(19) United States
(12) Patent Application Publication
(10) Pub. No.: US 2014/0162991 A1
(43) Pub. Date: Jun. 12, 2014

(54) TESTOSTERONE-ANASTROZOLE (T + A) COMBINATION IMPLANTS IN THE NEO-ADJUVANT, LOCAL AND SYSTEMIC THERAPY OF NEWLY DIAGNOSED, PREVIOUSLY UNTREATED BREAST CANCER

(71) Applicant: Rebecca L. Glaser, Dayton, OH (US)

(72) Inventor: Rebecca L. Glaser, Dayton, OH (US)

(21) Appl. No.: 14/090,443

(22) Filed: Nov. 26, 2013

Related U.S. Application Data
(60) Provisional application No. 61/731,100, filed on Nov. 29, 2012.

(51) Int. Cl. A61K 31/568; A61K 31/4196

(52) U.S. Cl. CPC A61K 31/568; A61K 31/4196
USPC 514/171

(57) ABSTRACT

A method for treating breast cancer in a patient is disclosed. The method includes locating a tumor in the breast, implanting into the tumor a first implant containing testosterone or an ester thereof and an aromatase inhibitor, and implanting a second implant containing testosterone or an ester thereof and an aromatase inhibitor into breast tissue adjacent to the tumor.

This application claims priority to U.S. Provisional Patent Application 61/731,100, filed on Nov. 29, 2012, and entitled TESTOSTERONE-ANASTROZOLE (T+A) COMBINATION IMPLANTS IN THE NEO-ADJUVANT, LOCAL AND SYSTEMIC THERAPY OF NEWLY DIAGNOSED, PREVIOUSLY UNTREATED BREAST CANCER, the entire contents of which is incorporated by reference herein.

### TECHNICAL FIELD

The present application relates to cancer treatment methods. More particularly, the present application relates to breast cancer treatment methods to provide differential dosing at the tumor site for local control of breast cancer.

### BACKGROUND

Testosterones biological effect at the androgen receptor inhibits the growth of breast cancer cells. However, testosterone may be aromatized to estradiol, which stimulates breast cancer due to the overexpression of aromatase in the cells in and around breast cancer tumors. Anastrozole inhibits the aromatization of testosterone to estradiol, which in turn prevents stimulation of breast tissue and breast cancer cells. Higher doses of therapy, e.g., testosterone and anastrozole, are needed locally at the cancer site in the breast to ensure efficacy, which is dose-dependent, while lower doses/levels are preferred systemically to avoid toxicities, which are similarly dose-dependent, but at the systemic level.

Other neo-adjuvant chemo and hormonal therapies rely on providing high doses of medication systemically, through oral or intravenous delivery methods/routes, in order to provide the adequate dose locally to the breast tissue/tumor site. Side effects of therapy are dose dependent, so high systematic dosing results in greater systemic side effects and toxicities, including toxicities specific to oral delivery.

### SUMMARY

The disclosed therapeutic method for treating a breast tumor provides a significantly higher dose of testosterone, or an ester thereof, with anastrozole (or another aromatase inhibitor) directly to the tumor and/or the tissue adjacent to the tumor (local therapy), while simultaneously providing lower systemic levels of therapy, thereby minimizing systematic side effects. The disclosed method allows the delivery of testosterone or ester with an aromatase inhibitor combined in a pellet implant providing adequate levels of testosterone both locally and systemically without elevating (stimulatory) estradiol levels. The combination pellet is implanted directly into the breast tissue at the tumor site, allowing markedly higher levels of therapy for local control of disease while providing adequate, but lower systemic levels of testosterone. U.S. Publication 2010/0144687 is incorporated herein by reference with respect to implants suitable for use with the disclosed therapeutic method. Research studies pertaining to the disclosed method include: Rebecca L. Glaser & Constantine Dimitrakakis, Reduced Breast Cancer Incidence in Women Treated with Subcutaneous Testosterone, or Testosterone with Anastrozole: A Prospective, Observational Study. MATURITAS, Vol. 76, Issue 4, pp. 342-349 (2013), and Rebecca L. Glaser & Constantine Dimitrakakis, Case Report: Rapid Response of Breast Cancer to Neoadjuvant Intramammary Testosterone-Anastrozole Therapy: Neoadjuvant Hormone Therapy in Breast Cancer, MENOPAUSE: THE JOURNAL OF THE NORTH AMERICAN MENOPAUSE SOCIETY, Vol. 21, No. 6 (2013), each of which are incorporated by reference herein in their entireties.

The implant is composed of testosterone or an ester thereof (T) with an aromatase inhibitor (A) such as anastrozole. The T+A implants can be inserted directly into a breast cancer/tumor (intra-tumoral) and/or the adjacent breast tissue (i.e. intramammary or peri-tumoral). The intramammary location is particularly unique in that it allows differential dosing. This is in direct contrast with neo-adjuvant therapy by other delivery methods including oral or intravenous, where higher systemic levels/doses are necessary to get adequate levels/dosing to the breast tissue/tumor site, resulting in greater systemic side effects and toxicities and less effective local therapy. The implant administered as disclosed herein provides local control of breast cancer disease by local/peritumoral delivery of high doses of T+A therapy as well as systemic control of disease with lower dose systemic delivery.

Testosterone is beneficial for immune function, and also treats many systemic symptoms of hormone deficiency, side effects of cancer therapies, as well as the breast cancer itself The sustained release combination implant assures continuous, simultaneous delivery and simultaneous absorption of both active ingredients locally at the tumor site while maintaining consistent, therapeutic systemic levels of T without elevating estradiol. It has been found that pharmacologic doses of testosterone produce a physiologic (therapeutic) effect, and such doses are not associated with adverse events. The only expected androgenic side effect in our patients has been a slight increase in facial hair. The safety of a 180 mg T implant dose has also been confirmed in historic studies, where doses from 150-225 mg were routinely prescribed, and doses up to 800-1800 mg were safely used long-term to treat metastatic breast cancer and female to male transgender patients.

The disclosed method may provide a number of benefits. As a neo-adjuvant therapy, administering the T+A implant may reduce tumor size and burden, assist in determining the patient's in vivo sensitivity to therapy (i.e. to identify responders vs. non-responders early in the therapeutic process), or avoid the need for the patient to undergo surgery and/or radiation. Even where surgical intervention is required, the reduction in tumor volume effected by the disclosed method may increase the efficacy of breast-conserving surgery, with improved cosmetic oncoplastic results. In addition, the disclosed method can provide local therapy for recurrent disease (cancer) in a previously irradiated breast where the only other option for local control of disease would be a completion mastectomy.

Also, the local, intra-mammary and intra-tumoral delivery methodology provides higher doses of T+A at the tumor site, providing greater local efficacy, while decreasing systemic absorption/dosing, resulting in lower systemic toxicities. The active ingredients are both 100% bio-available. In particular, it is the combination (T+A) implant, which facilitates steady state release of both T+A, providing continuous delivery of both active ingredients simultaneously to the tumor site as well as continuous systemic delivery (with differential dosing). The combination of both active ingredients in a single implant assures simultaneous release of both active ingredients (i.e. T+A) in the correct/fixed dose proportions, as well as simultaneous absorption of both active ingredients in the correct/fixed dose proportions.

Subcutaneous delivery of the fat-soluble testosterone and the moderately aqueous-soluble anastrozole together as a singular implant provides additional benefits as compared to delivering T and A separately using a combination of independent delivery methods (for example orally, or via separate testosterone anastrozole implants). Using a single implant to ensure both consistent release relates and consistent absorption rates of both drugs simultaneously is critical, because the pharmacokinetic/pharmacodynamic (PK/PD) characteristics of fat-soluble and aqueous-soluble drugs generally differ, which complicates coordination of the dosages. Thus, mixing the active ingredients (T+A) together in a combination implant and delivering it directly to the tumor eliminates the concern of differential release and absorption, thereby providing steady state release and absorption of both drugs simultaneously as the implant erodes in the breast, with higher levels of both active ingredients being delivered directly to the tumor, as compared to known delivery methods. The effectiveness of the T+A implant in facilitating linear release of both active ingredients has been demonstrated in vitro by accelerated dissolution analysis performed in a fat soluble medium (see FIG. 9). The linear release of the combination of A with T is identical to T alone, assuring clinical effective release and subsequent simultaneous absorption of both active ingredients in vivo, both at the tumor site and systemically.

Still further, the implant may provide long-acting, controlled, sustained release for up to three or more months of therapy with a single insertion. The implant does not require removal, as it may be completely absorbable. The combined implant provides steady state release and subsequent absorption thereof of both testosterone and anastrozole at lower systemic levels, 24 hours per day, which most closely approximates endogenous release of testosterone. Thus, the continuous, simultaneous release of substantially lower systemic levels, yet clinically proven effective doses of each drug, allows for a physiological/biological effective therapy with a significantly reduced potential for side effects.

Unlike oral delivery, parenteral subcutaneous and intramammary delivery of the implant avoids the gastrointestinal tract, thereby minimizing or avoiding complications and side effects of oral delivery of both testosterone and anastrozole including, but not limited to increased clotting, deep venous thrombosis, pulmonary embolisms, nausea, indigestion, elevated liver enzymes and specifically hepatic toxicity.

Administering T+A through the combination implant also allows for a markedly reduced dose of anastrozole (e.g., about 0.04 to about 0.12 mg subcutaneously, daily) as compared to a conventional dosage of 1 mg orally per day. This may reduce the occurrence of dose-dependent side effects of anastrozole intake, including hot flashes, pain, depression, bone fractures, and the like.

Patient compliance issues common to other delivery methods, such as oral delivery, are also avoided; once the T+A implant is implanted, no further action on the part of the patient is required to maintain the proper dosage. Notably, up to 40% of patients discontinue oral aromatase inhibitors due to side effects of therapy. With the combination T+A implant, compliance would necessarily be 100% of both active ingredients, thus assuring continuous highly effective local and systemic therapy.

The combination T+A implant is also cost-effective, providing about two to three months of therapy with a single, minimally invasive, minor procedure at a current cost substantially less than surgery, radiation and other hormonal and chemo therapies. This therapy may be suitable for use in conjunction with other endocrine therapies such as tamoxifen, or sulfatase inhibitors, which prevent hydrolysis of estrone sulfate and dehydroepiandrosterone sulfate (DHEAS), both of which can be reduced to steroids with estrogenic properties in a low estrogen environment.

In one aspect, a method for treating breast cancer in a patient is disclosed. The method includes locating a tumor in the breast, implanting into the tumor a first implant containing testosterone or an ester thereof and an aromatase inhibitor, and implanting a second implant containing testosterone or an ester thereof and an aromatase inhibitor into breast tissue adjacent to the tumor.

Other aspects of the disclosed structure and method will become apparent from the following description, the accompanying drawings, and the appended claims.

### BRIEF DESCRIPTION OF THE DRAWINGS

FIG. 1 depicts an incision and T+A implant locations for a breast cancer tumor (patient #1) according to the disclosed method;

FIG. 2 shows a series of mammograms from a case study of a patient (#1) treated in accordance with the disclosed method;

FIG. 3 shows a series of ultrasounds from patient #1 of the case study of FIG. 2;

FIG. 4 graphically depicts the decrease in tumor size of patient #1 of the case study of FIG. 2;

FIG. 5 is a table from a second case study (patient #2) that indicates the decrease in tumor size of a second patient treated in accordance with the disclosed method;

FIG. 6 graphically depicts the decrease in tumor size of patient #2 of the case study of FIG. 5;

FIGS. 7 (before therapy) and 8 (following therapy) show a series of mammograms from a third patient treated with intramammary/peritumoral placement of three T+A implants; and

FIG. 9 presents a graph comparing the near identical linear release characteristics of a T+A implant as compared to T alone.

### DETAILED DESCRIPTION

The following detailed description will illustrate the general principles of the invention, examples of which are additionally illustrated in the accompanying figures.

The inventive method involves locating a tumor in a breast, implanting into the tumor an implant, i.e., “pellet,” containing testosterone or an ester thereof and an aromatase inhibitor, and implanting an additional implant containing testosterone or an ester thereof and an aromatase inhibitor into breast tissue adjacent to the tumor. Alternately, the method may involve implanting a T+A implant solely into the breast tissue adjacent to the tumor. The method may further include surgical excision of any remaining portion of the tumor post-therapy. The method may be particularly useful for patients with AR (Androgen Receptor) positive tumors. In addition, the method may be particularly useful in “triple negative,” i.e., ER negative, PR negative, Her 2 negative, tumors for which other hormonal therapies are not indicated or ineffective.

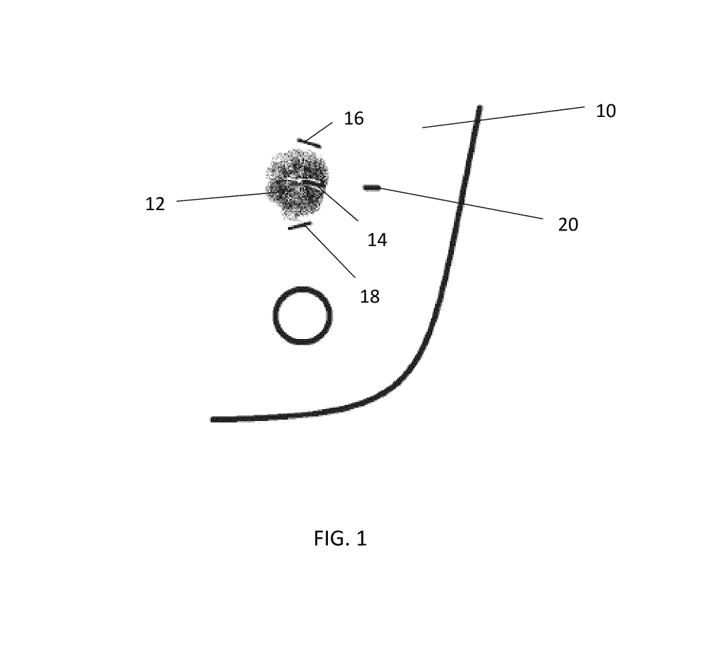

Referring to FIG. 1, a breast 10 is shown undergoing treatment according to one embodiment of the combination implant therapeutic method, where the breast 10 has a tumor 12 therein. In the depicted embodiment, three implants 14, 16, 18 are implanted intra-mammary/peritumoral and/or intra-tumorally into the breast 10 via an incision 20. Implant 14 is placed intra-tumorally, implant 16 is placed intra-mammary, superior to the tumor 12, and implant 18 is placed intra-mammary, inferior to the tumor 12. Alternately, implant 14 could be placed as a third intramammary implant, for example anterior to the tumor 12.

The implant is typically made up of the active ingredients, namely, testosterone or ester with an aromatase inhibitor such as, but not limited to, anastrozole, letrozole, or exemestane. In one embodiment, an implant is composed of the active ingredients testosterone and anastrozole. In one embodiment the implant is completely absorable and may consist of the active ingredients, however, to improve the integrity of the pellet and facilitate its manufacture, in one embodiment the active ingredients are used with excipients such as a lubricant and/or a binder. In one embodiment the lubricant is stearic acid. In one embodiment the binder is povidone. In one embodiment, the excipients make up less than about 20%, more particularly less than about 10%, and still more particularly less than about 5% of the composition. In one embodiment, these ingredients are formed into an implant such as a cylindrical pellet. The implant may be formed to any appropriate size, but for convenience of implanting using a standard size trocar, the implant may measure approximately 3.1 mm (diameter) by about 4.0 to about 10.0 mm (height), and in one embodiment approximately 3.1 mm (diameter) by about 6.4 mm (height). The implant is packaged and sterilized.

In one embodiment the implant contains the testosterone and the aromatization inhibitor in a weight ratio of about 5:2 to about 30:1 and, more particularly in a ratio of about 15:1 to about 25:1 and still more particularly about 10:1 to 20:1. For example, in one instance the implant includes about 60 mg testosterone and about 6 mg inhibitor (10:1), and in another instance the implant contains about 60 mg testosterone and about 4 mg inhibitor (15:1). In another instance the implant contains about 20 to 90 mg testosterone and about 2 to 9 mg aromatase inhibitor. In some embodiments, the dosage supplied by the implant is delivered over a period of at least about 30 days (about one month), or over a period of at least about 60 days (about two months) or over a period of at least about 90 days (about three months), or over a period of at least 120 days (about 4 months)

For intra-mammary, peri-tumoral implantation, the implant may fit through a standard size, 3.2 mm pellet implanter (i.e. a trocar/cannula), such that the implant can be inserted through a small incision, for example about 5 mm, into subcutaneous tissue of the breast, and/or deeper into the breast tissue; for example, directly into the breast tissue in an area surrounding a breast cancer tumor. In one embodiment, a single implant may be used. In another embodiment, two or more implants, for example up to about six implants, may be placed into the tissue surrounding the tumor. The implants may be placed adjacent to the tumor, for example up to about 2 cm, and in one embodiment up to about 1 cm, from the tumor in any direction, including but not limited to superior, inferior, anterior, posterior, medial and/or lateral placement relative to the tumor. In the case of diffuse disease, e.g., multifocal ductal carcinoma in situ or multifocal invasive carcinomas, the implants may be placed throughout the breast or throughout a quadrant of the breast.

For intra-tumoral implantation, the implant or multiple implants can be inserted through the trocar or core biopsy needle (e.g., MAMMOTOME®) directly into the tumor. In one embodiment, a single implant may be used. In another embodiment, depending on the size of the tumor, two or more implants, for example up to about six implants, may be placed directly into the tumor. In addition, the implants may be place in the axillary area or directly posterior to the nipple areolar complex.

The invention is illustrated by the following non-limiting examples:

### EXAMPLE 1

Three 3.1×6.1 mm implants 14, 16, 18, each containing 60 mg of testosterone combined with 4 mg of anastrozole are placed such that one is superior to the tumor 12, one is inferior to the tumor 12, and one is placed into the breast cancer tumor (see FIG. 1).

Total Dose: 180 mg Testosterone, 12 mg Anastrozole composed of three combination implants each containing, T 60 mg+A 4 mg, which may be re-dosed/implanted every 3 months.

Procedure: Three, 3.1×6.1 mm T 60 mg-A 4 mg pellet implants will be placed intra-mammary; into the tumor and surrounding tissue at the time of the core biopsy. A lateral incision may be used. The T-A implant may be placed prior to, or any time following the core biopsy. However, the core biopsy cannula should not be used for the two T-A pellets being placed into non-cancerous tissue, i.e., superior and inferior to the tumor. It may be used for the implant 14, which is to be placed directly into the tumor.

The first pellet implant (60T+4A) will be placed approximately 1 cm superior to the tumor using the disposable trocar inserted through a lateral incision. The trocar will be re-directed and second pellet implant (60T+4A) will be placed approximately 1 cm inferior to the tumor. The third implant is to be placed into the center of the tumor. In one embodiment, this may be placed through the biopsy cannula directly following the core biopsy. Again, because of the risk of contamination, the disposable trocar should not be used to place the implant into the tumor PRIOR to placing the pellet superior and inferior to the tumor in non-cancerous tissue. The disposable trocar may be used to insert the T-A implant into the center of the tumor AFTER the other pellets are inserted superiorly and inferiorly.

### EXAMPLE 2

Three 3.1×6.1 mm implants 14, 16, 18, each containing 60 mg of testosterone combined with 4 mg of anastrozole are placed such that one is superior to the tumor 12, one is inferior to the tumor 12, and one is placed anterior to the tumor.

Total Dose: 180 mg Testosterone, 12 mg Anastrozole composed of three combination implants each containing, T 60 mg+A 4 mg, which may be re-dosed/implanted every three months.

Procedure: Three, 3.1×6.1 mm T 60 mg−A 4 mg pellet implants will be placed intra-mammary/peritumoral, surrounding the tumor at the time of the core biopsy or alternatively, any time following the diagnosis of breast cancer. A lateral incision may be used. The T-A implant may be placed prior to, or anytime following, the core biopsy. The core biopsy cannula used to remove cancerous tissue for histological diagnosis should not be used for the peritumoral insertion of T-A pellets because, theoretically, “seeding” of tumor cells into non-cancerous tissue could occur. A sterile, disposable trocar may be used for intramammary/peritumoral placement of T-A pellets.

The first pellet implant (60T+4A) will be placed approximately 1 cm superior to the tumor using the disposable trocar inserted through a lateral incision. The trocar will be re-directed and second pellet implant (60T+4A) will be placed approximately 1 cm inferior to the tumor. The third implant (60T+4A) will be placed anterior to the tumor. In another scenario the implants could be placed posteriorly, lateral, or medial to the tumor.

### CLINICAL DATA

The efficacy and safety of the subcutaneous implantation of a T+A implant as a neo-adjuvant, therapeutic method for the treatment of breast cancer is illustrated by the following case presentations:

Case 1: 90 year old, G2P2 female with a history of menarche at age 14, natural menopause at age 43, and a family history of a grandmother with breast cancer, that presented with a 2.4 cm×2.3×1.7 cm tumor (i.e. tumor volume of 5.12 cc) in the subareolar 3 o'clock position. There was a slight thickening in the left breast at the 3 o'clock position at the areolar border, extending under the nipple-areolar complex. There was no palpable axillary adenopathy and no skin or nipple changes. An ultrasound guided core biopsy revealed a grade 2 infiltrating ductal carcinoma, Estrogen Receptor (ER) positive, Progesterone Receptor (PR) positive, Androgen Receptor (AR) positive, and HER2 negative. Following the ultrasound, the patient began therapy consisting of 20 mg of tamoxifen daily, but tamoxifen therapy was discontinued after implantation of the T+A pellets.

Through a 5 mm lateral incision, three compounded/prototype 60 mg T+4 mg A pellets were implanted into the breast tissue surrounding the tumor approximately 1 cm superior to, 1 cm inferior to, and anterior to the subareolar tumor through a disposable trocar. Follow up examination of the left breast two weeks after intramammary T+A pellet implantation revealed a marked decrease in the size of the tumor on physical exam and office ultrasound; the periareolar thickening was no longer palpable. By week 4, the patient's (previously unreported) left breast pain had subsided.

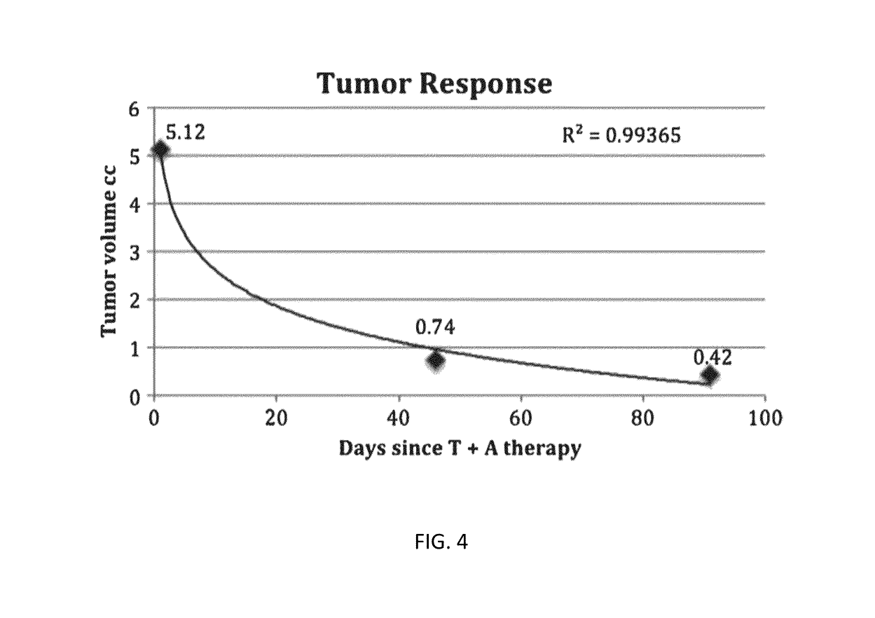

Forty-six days following the intra-mammary T+A therapy, a follow-up left breast ultrasound revealed a significant decrease in the size of the tumor mass. On ultrasound, the tumor measured 1.6×1.1×0.8 cm: a tumor volume of 0.74 cc, indicating a 7-fold reduction in tumor volume as compared to the initial 5.13 cc (FIG. 4).

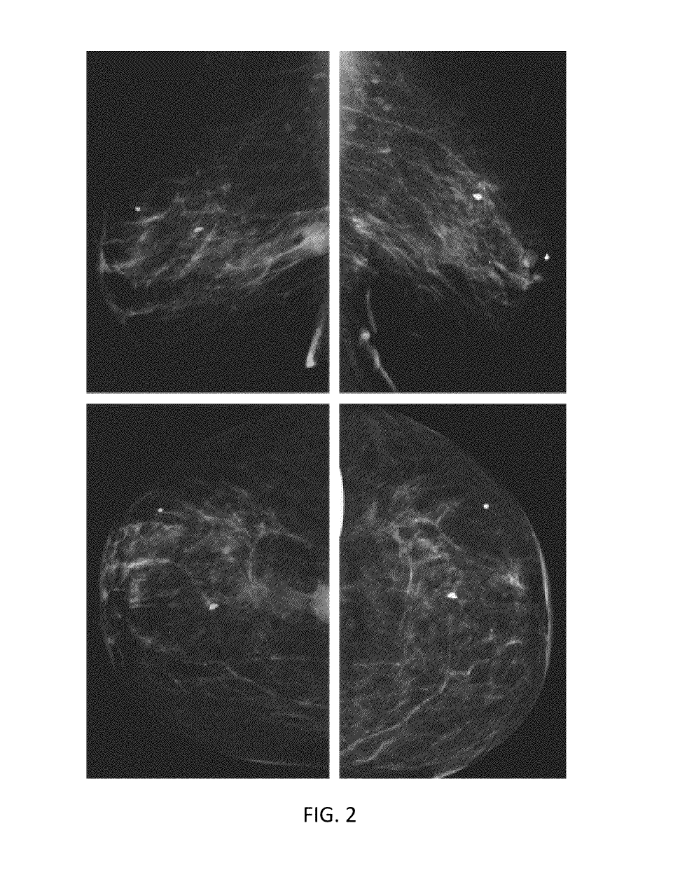

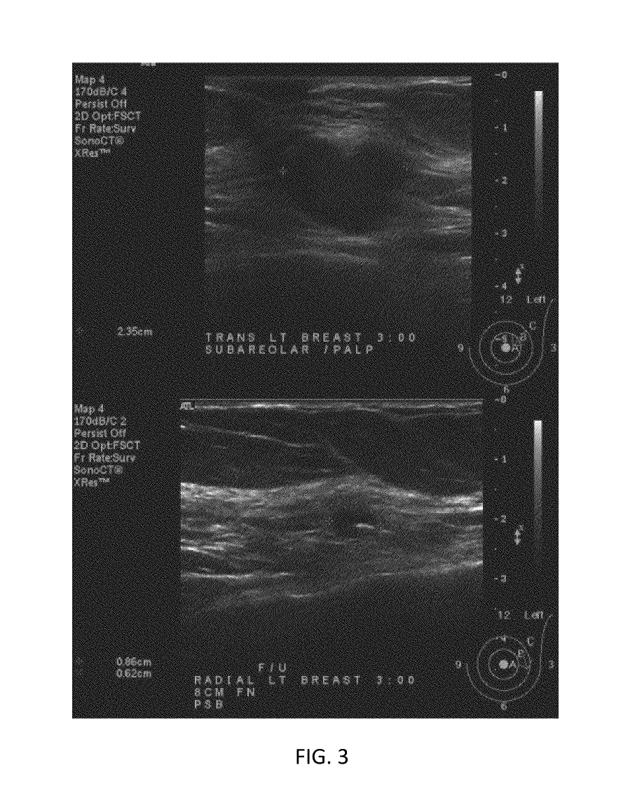

Two days later, three additional T 60 mg+A 4 mg implants (i.e., a total dose of 180 mg T+12 mg A) were again placed peritumoral in the left breast. A follow up mammogram and ultrasound were performed at week 13, revealing further reduction in tumor size to 1.5×0.8×0.6 cm: a tumor volume of 0.42 cc, indicating a 12-fold reduction from the original tumor measurements (FIGS. 2-4). This equates to a 2.78% decrease per day (following therapy) and a half-life of 3 days. FIG. 2 shows comparative mammograms comparing the pre-treatment tumor with the tumor after 13 weeks (top left: mediolateral oblique (MLO) view prior to treatment; top right: MLO view after 13 weeks; bottom left: craniocaudal (CC) view prior to treatment; bottom right: CC view after 13 weeks). FIG. 3 shows an ultrasound pre-treatment (top) as compared to post-treatment (bottom). The logarithmic response of the carcinoma to T+A therapy is evidenced by a R²>0.99 (FIG. 4). By week 30, tumor volume measured 0.157 cc, a 33-fold reduction, i.e., 3% of the original tumor volume. Of significance, a follow up core biopsy downgraded the tumor from nuclear grade 2 to nuclear gradel.

In addition, many of the patient's systemic symptoms including memory loss, physical fatigue, urinary incontinence, sleep disturbance, depression, and pain were improved on the systemic T therapy. Adequate serum levels of T, without elevation of estradiol (E2), was confirmed on day 7 post-insertion (T 473 ng/dl, E2<5 pg/ml), day 46 post-insertion (T 366 ng/dl, E2<5 pg/ml), and again on day 7 following the second intra-mammary insertion procedure (T 345 ng/dl, E2<5 pg/ml). Interestingly, the patient was able to discontinue several medications in addition to tamoxifen, including duloxetine Hcl, lisinopril, and atorvastatin. There have been no adverse drug effects with the therapy and no evidence of systemic disease on therapy.

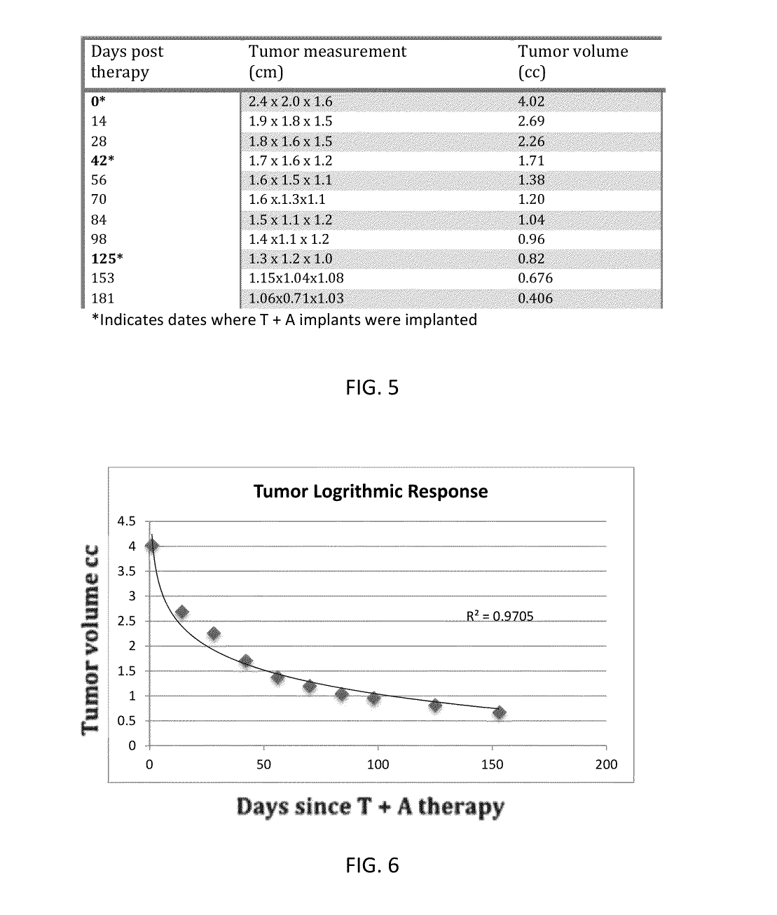

Case 2: 80 year old female who presented with a large, 3 cm, palpable, firm right breast mass and palpable axillary adenopathy. A core biopsy revealed an infiltrating ductal carcinoma ER positive, PR positive, Androgen receptor (AR) positive with axillary metastasis, i.e., lymph node involvement. Three T 60 mg+A 4 mg implants were placed in the breast (intramammary) surrounding the tumor (peritumoral). FIG. 5 indicates the reduction in tumor volume over time, and FIG. 6 shows the logarithmic response of the carcinoma over time. Over the course of about six months, the tumor volume decreased from 4.02 cc to 0.406 cc; showing reduction in tumor size at a rate of 1.87% per day, with a half life of 37 days and a logarithmic decline in tumor volume, R²=0.96. This patient's palpable adenopathy is also responded to therapy.

Four additional patients (#3, #4, #5 and #6) with invasive ductal or lobular breast cancers have been treated with the combination T 60 mg+A 4 mg implants. Each patient has received three combination implants for a total dose of 180 mg T and 12 mg A, placed in the breast tissue surrounding their tumors. Remarkably, all four additional patients have responded to this therapy as demonstrated on follow up clinical exams, serial ultrasounds, and mammograms. Patients include both premenopausal women and postmenopausal women. Tumors include invasive ductal carcinomas and invasive lobular carcinomas, as well as multifocal and multi-quadrant disease.

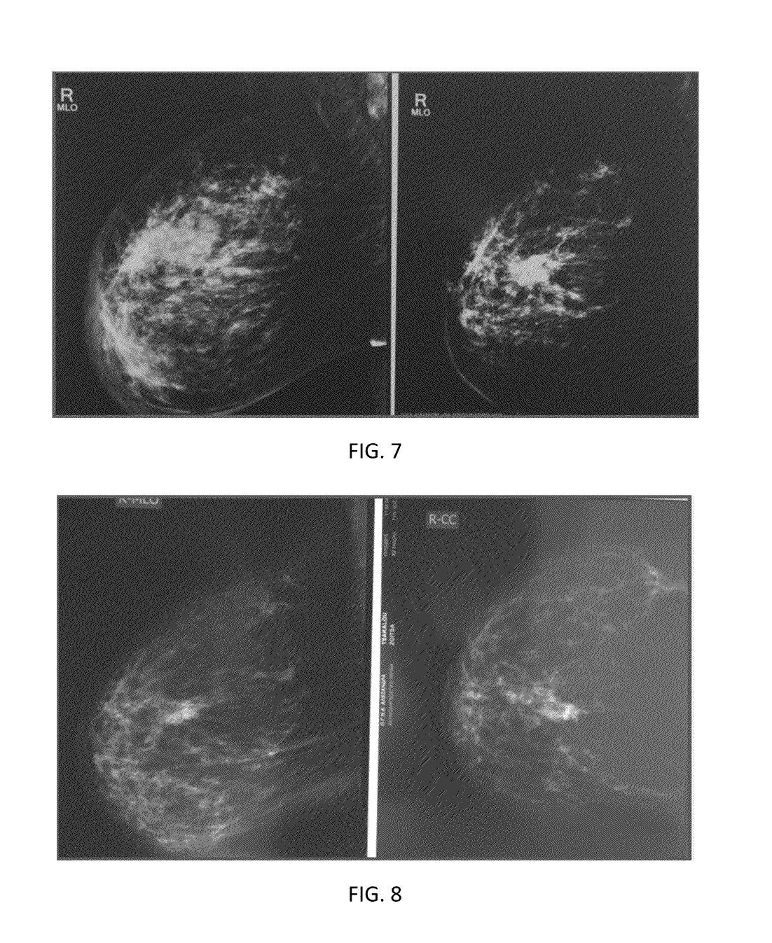

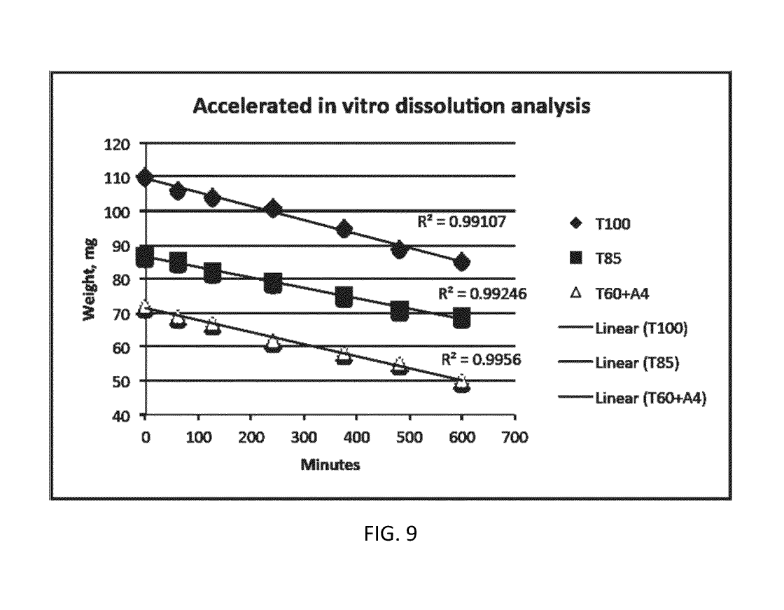

Notably, patient #3's, tumor responded rapidly to neoadjuvant intramammary T+A implant therapy, as did her visible axillary lymph nodes, which are no longer visible on mammogram (compare FIG. 7, MLO and CC views prior to treatment, with FIG. 8, MLO and CC views post-treatment) and were no longer palpable on clinical exam. In addition, patient#3 feels great on the systemic levels of testosterone and has no adverse side effects from therapy. Furthermore, there is no evidence of systemic disease.

While the invention has been illustrated by several expressions of several embodiments, and enablements, and applications, thereof, the invention is defined by the appended claims and is not limited to the specific examples. Numerous variations, modifications, and substitutions are possible without departing from the scope of the invention as defined in the appended claims. It will be understood that the foregoing description is provided by way of example, and that other modifications may occur to those skilled in the art without departing from the scope and spirit of the appended claims.

What is claimed is:

1. A method for treating breast cancer in a patient comprising the steps of:
locating a tumor in the breast;
implanting into the tumor a first implant containing testosterone or an ester thereof and an aromatase inhibitor, and
implanting a second implant containing testosterone or an ester thereof and an aromatase inhibitor into breast tissue adjacent to the tumor.

2. The method of claim 1, wherein the first implant and the second implant contain different amounts of testosterone and/or different amounts of aromatase inhibitor.

3. The method of claim 1, wherein the first implant and the second implant contain the same amounts of amounts of testosterone and/or the same or different amounts of aromatase inhibitor.

4. The method of claim 1, wherein two or more second implants are implanted in the breast tissue adjacent to the tumor.

5. The method of claim 4, wherein the second implants are approximately equally spaced around the tumor.

6. The method of claim 1, wherein one or more first implants is/are implanted in the tumor.

7. The method of claim 1, wherein the weight ratio of the testosterone to the aromatase inhibitor in each of the first and second implants is from about 5:2 to about 30:1, and the testosterone or ester dosage is about 30 mg to about 200 mg delivered over a period of at least about 30 days, and wherein the aromatase inhibitor dosage is about 1 to about 90 mg delivered over a period of at least about 30 days.

8. The method of claim 1, wherein at least one of the first implant and the second implant contains about 60 mg testosterone and about 4 mg aromatase inhibitor.
